(19) United States
(12) Patent Application Publication
(10) Pub. No.: US 2013/0172962 A1
(43) Pub. Date: Jul. 4, 2013

(54) OPTICAL PROBE LED CHIP MODULE FOR BIOSTIMULATION AND METHOD OF MANUFACTURING THE SAME

(71) Applicants: Jin Dong Song, Seoul (KR); Ji Hyun Choi, Seongnam-si (KR); Ju Hyeon Lee, Seoul (KR)

(72) Inventors: Jin Dong Song, Seoul (KR); Ji Hyun Choi, Seongnam-si (KR); Ju Hyeon Lee, Seoul (KR)

(73) Assignee: Korea Institute of Science and Technology, Seoul (KR)

(21) Appl. No.: 13/412,695

(22) Filed: Mar. 6, 2012

(30) Foreign Application Priority Data
Jan. 2, 2012 (KR) 10-2010-0000141

(51) Int. Cl. A61N 5/06; H01R 43/00

(52) U.S. Cl. 607/90; 607/88; 29/825

(57) ABSTRACT

A probe-type light-emitting diode (LED) chip module for biostimulation includes: an LED chip, a substrate supporting the LED chip, an optical waveguide collecting light emitted from the LED chip; and an insulator coupling the substrate with the optical waveguide and providing insulation from outside. The optical waveguide includes: a body extending from one end facing the LED chip with a cylindrical shape; a variable layer having a diameter decreasing gradually from the other end of the body; and a probe extending from the end of the variable layer and having a diameter equivalent to that of an optical fiber. The probe-type LED chip module for biostimulation may be manufactured with a small size to have superior portability and applicability.

### CROSS-REFERENCE TO RELATED APPLICATIONS

This application claims priority under 35 U.S.C. §119 to Korean Patent Application No. 10-2012-0000141, filed on Jan. 2, 2012, in the Korean Intellectual Property Office, the disclosure of which is incorporated herein by reference in its entirety.

### BACKGROUND

1. Field

The present disclosure relates to a probe-type light-emitting diode (LED) chip module for biostimulation and a method for manufacturing the same. More particularly, the disclosure relates to a small-sized, probe-type LED-based chip module for biostimulation and a method for manufacturing the same.

2. Description of the Related Art

Electrical stimulation is applied to the living body, especially brain, to understand and treat cranial nerve diseases such as Parkinson's disease, depression, or the like. However, the site of electrical stimulation should be accurate in the cellular scale. And, an excessive intensity may lead to side effects such as dizziness or numbness.

In addition, electrical noise makes the detection of the brain signal difficult, and brain scan images cannot be obtained since the patient cannot enter a magnetic resonance imaging (MRI) instrument because a metal needle is used.

Accordingly, stimulation of the brain via optical stimulation rather than electrical stimulation is developed recently and is used for treatment of cranial nerve disease as well as improvement of functions of organs such as muscles and kidneys.

FIG. 1 and FIG. 2, extracted respectively from Aravanis A, et al. J. Neural Eng. September 2007; 4:S143-S156 and Zhang F, et al. Nature. Apr. 5 2007; 446:633-3, show a schematic of an existing system using a large-size laser and an optical fiber, change in signals from a laboratory mouse with wavelength of incident light, and operation principle thereof.

As seen from FIG. 1, a laser and an optical fiber are provided at specific portion of the brain of a laboratory mouse. For example, if there are ChR2 photoreceptors in the brain of the laboratory mouse, it can be seen from the graph of FIG. 2 that the brain wave of the mouse changes when the brain is exposed to blue light centered at about 425 nm.

However, since the optical stimulation adopts the method of transmitting optical signal by connecting the optical fiber to the large-size laser, a living organism is restricted a lot in activities. Accordingly, a small-sized optical stimulation device capable of replacing the optical stimulation device based on the large-size laser is needed.

### SUMMARY

The present disclosure is directed to providing a probe-type light-emitting diode (LED) chip module for biostimulation with superior portability and applicability.

The present disclosure is also directed to providing a method for easily manufacturing the probe-type LED chip module for biostimulation.

In one general aspect, the present disclosure provides a probe-type LED module for biostimulation including: an LED chip; a substrate supporting the LED chip; an optical waveguide collecting light emitted from the LED chip; and an insulator coupling the substrate with the optical waveguide and providing insulation from outside.

In an exemplary embodiment of the present disclosure, the optical waveguide may include: a body extending from one end facing the LED chip with a cylindrical shape; a variable layer having a diameter decreasing gradually from the other end of the body; and a probe extending from the end of the variable layer and having a diameter equivalent to that of an optical fiber.

In an exemplary embodiment of the present disclosure, the optical waveguide may be from an optical fiber preform. In that case, the optical waveguide may be formed from silica.

In an exemplary embodiment of the present disclosure, the optical waveguide may have a double cylindrical structure including: a core formed at the center of a length direction of the optical waveguide and transmitting light emitted from the LED chip; and a cladding surrounding the core.

In an exemplary embodiment of the present disclosure, the refractive index of the core of the optical waveguide may be higher than the refractive index of the cladding. In this case, germanium oxide (GeO₂) may be doped in the core of the optical waveguide.

In an exemplary embodiment of the present disclosure, the probe-type LED chip module for biostimulation may further include an electrode connector connected to an external power supply supplying power to the LED chip. In this case, the substrate may electrically connect the LED chip with the electrode connector.

In an exemplary embodiment of the present disclosure, the substrate may be a ceramic substrate including aluminum nitride (AlN), aluminum oxide (Al₂O₃), etc. or a printed circuit board (PCB).

In an exemplary embodiment of the present disclosure, the probe-type LED chip module for biostimulation may be used for the ChR2 receptor. In this case, the LED chip may include a gallium nitride (GaN)-based blue LED.

In an exemplary embodiment of the present disclosure, the LED chip may include a plurality of LEDs having different wavelength each other.

In an exemplary embodiment of the present disclosure, the probe-type LED chip module for biostimulation may further include an optical matching material between the substrate and the optical waveguide.

In an exemplary embodiment of the present disclosure, the insulator may be formed from a light-absorbing insulating material. In this case, the insulator may be formed from black epoxy.

In another general aspect, the present disclosure provides a method for manufacturing a probe-type LED chip module for biostimulation, including: extending an optical fiber preform whose refractive index at the center being higher than the refractive index at the periphery to form a cylinder-shaped intermediate preform; extending the intermediate preform with and without heating to form an optical waveguide; coupling the optical waveguide with a substrate on which an LED chip is mounted; and sealing the substrate and the optical waveguide with a light-absorbing insulator.

In an exemplary embodiment of the present disclosure, the step of forming the optical waveguide may include: extending the intermediate preform slowly with heating to form a variable layer having a diameter decreasing gradually from a portion of the intermediate preform; and extending the end of the variable layer quickly without heating to form a probe having a diameter equivalent to that of an optical fiber.

In an exemplary embodiment of the present disclosure, the step of forming the optical waveguide may be performed after fixing an end of the intermediate preform to a holder.

In an exemplary embodiment of the present disclosure, the step of coupling the optical waveguide with the substrate may include injecting an optical matching material between the substrate and the optical waveguide.

In an exemplary embodiment of the present disclosure, the step of coupling the optical waveguide with the substrate may further include mirror polishing the lower end of the optical waveguide.

In an exemplary embodiment of the present disclosure, the method for manufacturing a probe-type LED chip module for biostimulation may further include forming an electrode connector connected to an external power supply supplying power at the substrate.

The probe-type LED chip module for biostimulation according to the present disclosure can be manufactured into a small size since the LED is used. Therefore, since nerves can be stimulated without affecting the activities of a living organism, an optical stimulation device with superior portability and applicability can be provided. Furthermore, the LED-based device can be manufactured at low cost as a disposable device.

### BRIEF DESCRIPTION OF THE DRAWINGS

The above and other objects, features and advantages of the present disclosure will become apparent from the following description of certain exemplary embodiments given in conjunction with the accompanying drawings, in which:

FIG. 1 schematically shows an existing optical stimulation device using a large-size laser and an optical fiber;

FIG. 2 shows change in signals from a laboratory mouse when the optical stimulation device of FIG. 1 is used;

FIG. 3 is a cross-sectional view of a probe-type light-emitting diode (LED) chip module for biostimulation according to an embodiment of the present disclosure in a length direction;

FIG. 4 conceptually shows an example of using the probe-type LED chip module for biostimulation of FIG. 3;

FIG. 5 shows optical output of the probe-type LED chip module for biostimulation of FIG. 3;

FIG. 6 shows wavelength spectrum of the probe-type LED chip module for biostimulation of FIG. 3;

FIG. 7 schematically shows a system using the probe-type LED chip module for biostimulation of FIG. 3; and

FIG. 8 shows cross-sectional views illustrating a method for manufacturing the probe-type LED chip module for biostimulation of FIG. 3.

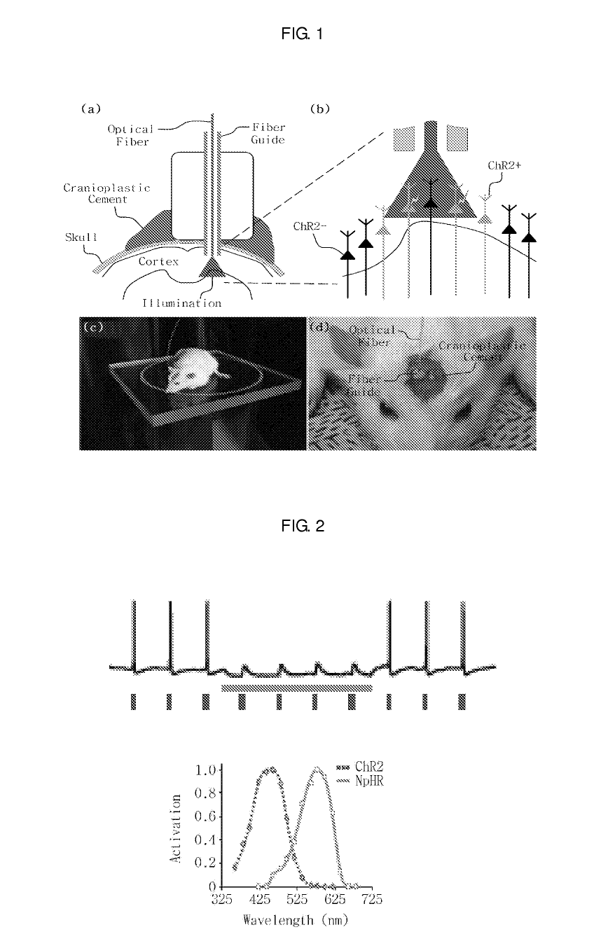

### DETAILED DESCRIPTION

Hereinafter, exemplary embodiments of a probe-type light-emitting diode (LED) chip module for biostimulation and a method for manufacturing the same will be described in detail with reference to the accompanying drawings.

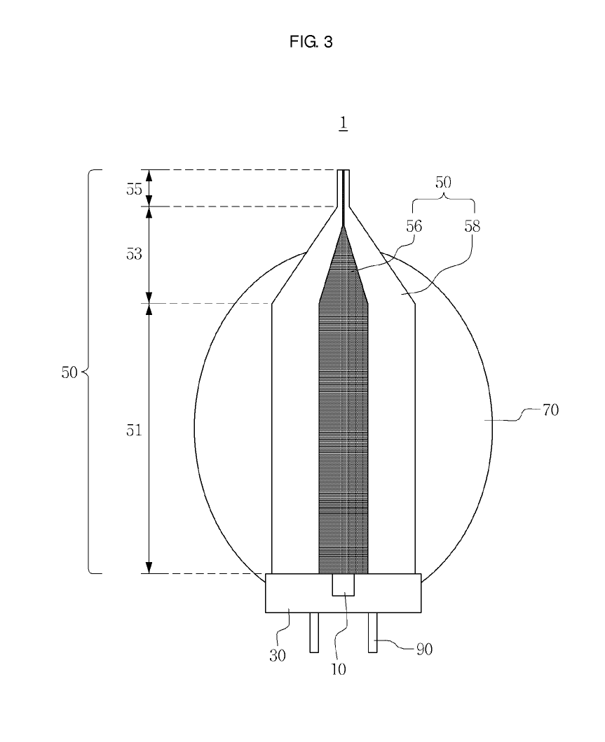

FIG. 3 is a cross-sectional view of a probe-type LED chip module for biostimulation according to an embodiment of the present disclosure in a length direction.

Referring to FIG. 3, a probe-type LED chip module 1 for biostimulation according to an embodiment of the present disclosure comprises an LED chip 10, a substrate 30 supporting the LED chip 10, an optical waveguide 50 collecting light emitted from the LED chip 10, and an insulator 70 coupling the substrate 30 with the optical waveguide 50 and providing optical/electrical/mechanical insulation from outside.

And, the probe-type LED chip module 1 for biostimulation may further comprise an electrode connector 90 connected to an external power supply supplying power to the LED chip 10.

The LED chip 10 may comprise one or more LEDs as a semiconductor comprising gallium (Ga), indium (In), aluminum (Al), nitrogen (N), phosphorus (P), arsenic (As), antimony (Sb), etc. The LEDs may have different wavelength each other and the particular different wavelength may be selectively used.

For example, when the probe-type LED chip module 1 for biostimulation is used for the ChR2 receptor, the LED chip 10 may comprise a gallium nitride (GaN)-based blue LED. However, the material, wavelength and output of the LED chip 10 may be selected according to the particular disease or disorder, without being limited thereto.

The substrate 30 is coupled with the optical waveguide 50, with the LED chip 10 mounted. The substrate 30 supports not only the LED chip 10 but also the optical waveguide 50, and supports the whole probe-type LED chip module 1 for biostimulation.

The substrate 30 may be formed from a material with high thermal conductivity so that the heat generated as the LED chip 10 emits light can be easily dissipated. And, an electrical passage is formed on the substrate 30 so as to supply the power applied to the electrode connector 90 to the LED chip 10.

For example, the substrate 30 may be a ceramic substrate comprising aluminum nitride (AlN), aluminum oxide (Al₂O₃), etc. Alternatively, when a high optical output is not required for the LED chip 10, the substrate 30 may be an inexpensive and commonly used printed circuit board (PCB).

The optical waveguide 50 collects the light emitted from the LED chip 10 and stimulates a living body. The optical waveguide 50 may be formed from an optical fiber preform, for example, silica. Alternatively, the optical waveguide 50 may be formed from various transparent materials such as acrylic resin if wavelength and output are compatible.

When viewed from a cross section, the optical waveguide 50 has a double cylindrical structure comprising a core 56 formed at the center of a length direction of the optical waveguide 50 and a cladding 58 surrounding the core 56. That is to say, the optical waveguide 50 has a modified double cylindrical structure.

The core 56 is formed from a material having a higher refractive index (e.g. germanium oxide (GeO₂)-doped silica) than that of the cladding 58. Accordingly, since the optical fiber preform has a structure similar to that of a single-mode or multi-mode optical fiber after it is finally extended, the light emitted from the LED chip 10 is transmitted through the core 56.

In order to increase the refractive index of the core 56, an impurity may be doped in the center portion of the optical waveguide 50. For example, the impurity may be germanium (Ge).

When viewed in the length direction, the optical waveguide 50 comprises a body 51 extending with a cylindrical shape, a variable layer 53 having a diameter decreasing gradually from the body 51, and a probe 55 extending from the end of the variable layer 53 and having a diameter equivalent to that of an optical fiber.

Of course, the body 51, the variable layer 53 and the probe 55 of the optical waveguide 50 have relatively higher refractive index and differ only in diameter. Since the optical waveguide 50 comprises the core 56 formed at the center and the cladding 58 surrounding the core 56, the cross section of the body 51, the variable layer 53 and the probe 55 of the optical waveguide 50 has a structure of concentric circles with different diameters.

One end of the cylinder-shaped body 51 faces the LED chip 10 and receives light from the LED chip 10. The body 51 extends with a cylindrical shape. For example, the body 51 may have a diameter from about 1 mm to about 5 mm and may have a length of about 5 mm or smaller. But, the diameter and the length of the body 51 may be set differently, without being limited thereto.

The variable layer 53 is formed to have a shape of a truncated cone with a diameter decreasing gradually from the other end of the body 51. The lower end of the variable layer 53 with a larger cross-sectional area is connected to the body 51 and the upper end of the variable layer 53 with a smaller cross-sectional area is connected to the probe 55.

Such shape of the variable layer 53 allows the light incident from the end of the body 51 with a larger diameter to be transmitted effectively to the probe 55 having a smaller diameter with low loss of light. For example, the variable layer 53 may have a length of about 5 mm or smaller and the upper end may have a diameter of about 100 μm. But, the diameter and the length of the variable layer 53 may be set differently, without being limited thereto.

The probe 55 extends from the upper end of the variable layer 53 with a shape of a probe. The probe 55 may have a diameter of an optical fiber. For example, the probe 55 may have a diameter of about 100 μm and a length of about 5 mm or longer. But, the diameter and the length of the probe 55 may be set differently, without being limited thereto.

Since the probe 55 has a diameter of about 100 μm and comprises the core 56 with high refractive index and the cladding 58 surrounding the core 56, it has a structure similar to that of an optical fiber.

When viewed in the length direction, the optical waveguide 50 has a shape of a sharpened pencil. The light emitted from the LED chip 10 is transmitted through the core 56 formed at the center of the optical waveguide 50 to the probe 55. The probe 55 having a structure similar to that of an optical fiber collects the light and stimulates the living body.

An optical matching material for optical matching may be further provided between the substrate 30 and the optical waveguide 50. The optical matching material may improve optical matching between the LED chip 10 and the optical waveguide 50. In order to improve the efficiency of optical matching between the optical waveguide 50 and the LED chip 10, the lower end of the optical waveguide 50 may be mirror polished.

The insulator 70 couples the substrate 30 with the optical waveguide 50 and, at the same time, provides optical/electrical/mechanical insulation for the probe-type LED chip module 1 for biostimulation from outside. That is to say, the insulator 70 mechanically couples the probe-type LED chip module 1 for biostimulation, provides electrical insulation and waterproofing, and prevents leakage of light.

The insulator 70 may seal the probe-type LED chip module 1 for biostimulation except for the portion where the probe-type LED chip module 1 for biostimulation is connected with outside. For example, the insulator 70 may surround the probe-type LED chip module 1 for biostimulation while partly exposing the probe 55 and the electrode connector 90.

For this, the insulator 70 is formed from a biologically unharmful, highly light-absorbing, waterproofing, and electrically insulating material. That is to say, the insulator 70 may be formed from a light-absorbing insulating material, for example, black epoxy.

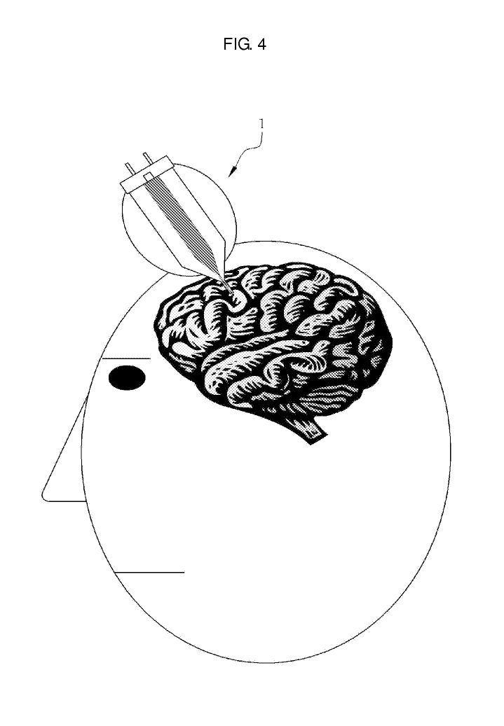

FIG. 4 conceptually shows an example of using the probe-type LED chip module for biostimulation of FIG. 3.

Referring to FIG. 4, the probe-type LED chip module 1 for biostimulation is mounted to a human brain to stimulate the brain. The probe 55 of the probe-type LED chip module 1 for biostimulation is in direct contact with the brain and transmits light thereto.

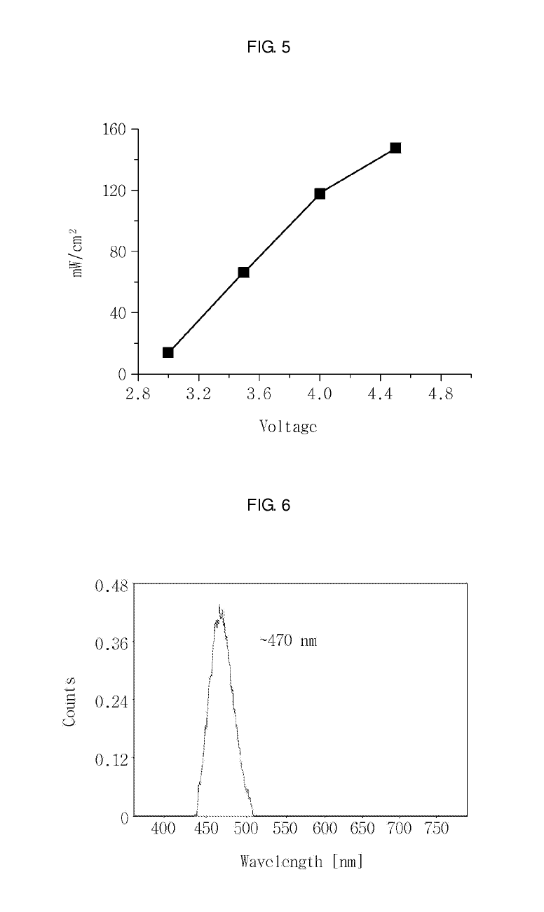

FIG. 5 shows optical output of the probe-type LED chip module for biostimulation of FIG. 3, and FIG. 6 shows wavelength spectrum of the probe-type LED chip module for biostimulation of FIG. 3.

FIG. 5 shows a result of measuring optical output using an integrating sphere while operating the probe-type LED chip module 1 for biostimulation with a continuous wave (CW). An output of about 150 mW/cm² can be obtained with a voltage of about 4.4 V.

This result is comparable to the optical output of the laser-based optical stimulation device at the end of the optical fiber and reveals that the small-sized optical stimulation device according to the present disclosure can replace the existing laser-based optical stimulation.

Referring to FIG. 6, it can be seen that the wavelength region of the LED chip 10 used in the probe-type LED chip module 1 for biostimulation according to the present disclosure is sufficient to activate the ChR2 receptor.

In the presented embodiment, the gallium nitride (GaN) LED of about 470 nm wavelength was used for optical stimulation of the ChR2 receptor in the brain. However, for treatment/improvement of other diseases/disorders at other parts of the body, an LED of different wavelength may be used to manufacture the probe-type LED chip module 1 for biostimulation.

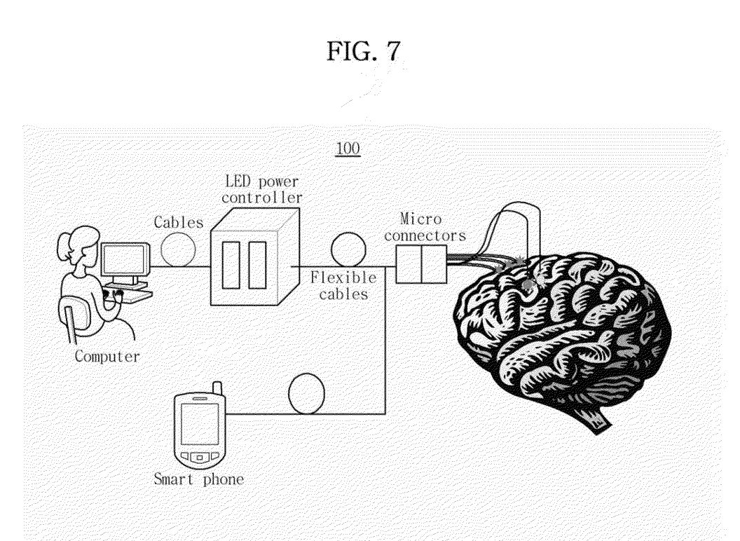

FIG. 7 schematically shows a system using the probe-type LED chip module for biostimulation of FIG. 3.

FIG. 7 shows a treatment system 100 based on optical stimulation using the probe-type LED chip module 1 for biostimulation according to the present disclosure. A controller may be used to interpret response to the optical stimulation. Also, a smart phone or similar mobile devices may be used to provide treatment based on the interpreted data.

The probe-type LED chip module 1 for biostimulation according to the present disclosure may, as an LED-based, small-sized optical stimulation device, replace the existing large-sized optical stimulation device to treat cranial nerve disease as well as to improve the functions of muscles, kidneys, or the like.

Also, the small size allows for free movement with little restriction in activities. In addition, since the cost of the optical stimulation device can be decreased, the device can be manufactured as a disposable device and the medical cost can be reduced.

Hereinafter, a method for manufacturing the probe-type LED chip module 1 for biostimulation according to an exemplary embodiment of the present disclosure will be described.

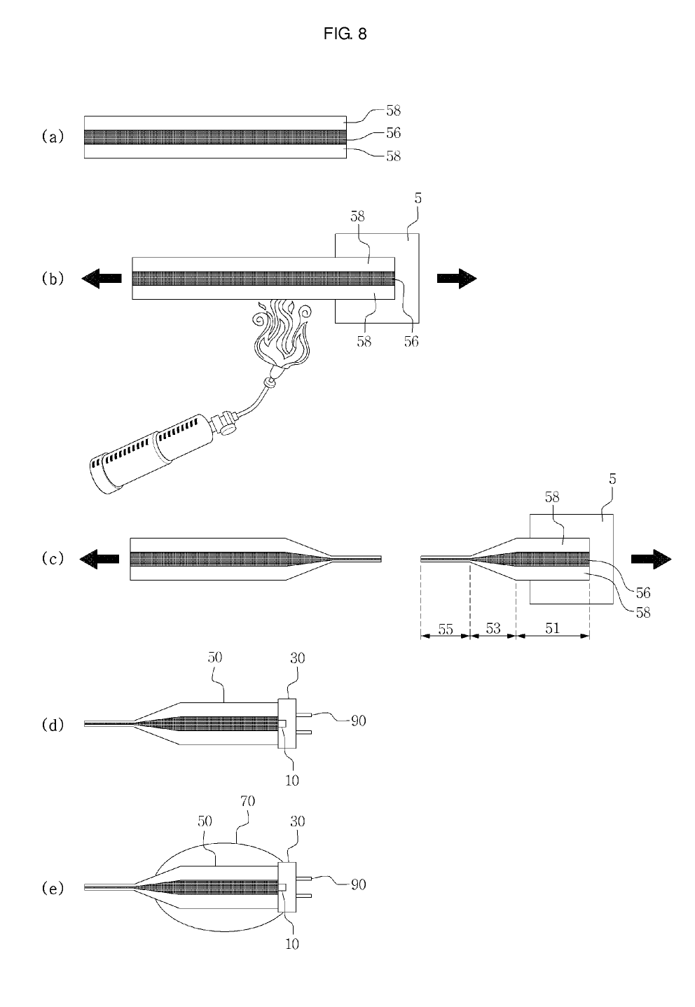

FIG. 8 shows cross-sectional views illustrating a method for manufacturing the probe-type LED chip module for biostimulation of FIG. 3.

Referring to FIG. 8 (a), an optical fiber preform whose refractive index at the center being higher than the refractive index at the periphery may be extended to form an intermediate preform. The optical fiber preform may be extended with heating. For example, the optical fiber preform may be formed from silica or a transparent material such as acrylic resin.

At the center of the optical fiber preform, the core 56 wherein an impurity is doped such that the refractive index at the center of the optical fiber preform is higher than that of the periphery is formed. That is to say, the optical fiber preform has a double cylindrical structure wherein the core 56 with a relatively higher refractive index is surrounded by the cladding 58 with a relatively lower refractive index.

For example, the impurity may be germanium oxide (GeO₂). Since the refractive index of the center of the optical fiber preform is higher than that of the periphery, light may be transmitted only through the center of the optical waveguide 50, i.e. the core 56.

The intermediate preform formed by extending the optical fiber preform may have a cylindrical shape. For example, it may have a diameter of about 1 mm to about 5 mm and a length of about 10 cm to about 20 cm. But, the diameter and the length of the intermediate preform may be set differently, without being limited thereto.

Referring to FIGS. 8 (b) and (c), the intermediate preform is extended with and without heating to form the optical waveguide 50.

In order to form the optical waveguide 50, the intermediate preform is first extended at low speed with heating, as shown in FIG. 8 (b). As the intermediate preform is extended with heating, the variable layer 53 with a shape of a truncated cone with a diameter decreasing gradually from a portion of the intermediate preform is formed.

The variable layer 53 may be extended until the end of the variable layer 53 has a diameter of an optical fiber. For example, the variable layer 53 may be extended until the length is about 5 mm or shorter and the diameter of the upper end with a smaller cross-sectional area is about 100 μm or smaller.

In this case, one end of the intermediate preform may be fixed to a holder 5. To reduce the length of the optical waveguide 50, the holder 5 should be located close to the heated portion. Accordingly, the holder 5 needs to be formed from a heat-resistant material.

When the length of the optical waveguide 50 is short, the length of the probe-type LED chip module 1 for biostimulation can be reduced.

Then, as seen from FIG. 8 (c), the end of the variable layer 53 is extended without heating. The end of the variable layer 53 is extended to form the probe 55 having a diameter of an optical fiber.

For example, the probe 55 may be extended until the diameter is about 100 μm or smaller and the length is about 5 mm or longer. But, the diameter and the length of the variable layer 53 and the probe 55 may be set differently, without being limited thereto.

Referring to FIG. 8 (d), the optical waveguide 50 is coupled with the substrate 30 on which the LED chip 10 is mounted. The side of the optical waveguide 50 coupled with the substrate 30 may be polished such that the light emitted from the LED chip 10 may pass through the optical waveguide 50 more easily.

An optical matching material for optical matching may be further provided between the substrate 30 and the optical waveguide 50. The optical matching material may improve optical matching between the LED chip 10 and the optical waveguide 50. The lower end of the optical waveguide 50 may be, for example, mirror polished in order to improve the efficiency of optical matching between the LED chip 10 and the optical waveguide 50 by reducing reflectivity.

The LED chip 10 may comprise one or more LEDs. For example, it may comprise a gallium nitride (GaN)-based blue LED. However, another LED of different wavelength may be used without being limited thereto according to the particular disease or disorder.

An electrical passage is formed on the substrate 30 so as to supply power from an external power supply to the LED chip 10. For example, the substrate 30 may be a ceramic substrate comprising aluminum nitride (AlN), aluminum oxide (Al₂O₃), etc. Alternatively, when a high optical output is not required for the LED chip 10, the substrate 30 may be an inexpensive and commonly used printed circuit board (PCB).

The electrode connector 90 connected to the external power supply may be further formed on the substrate 30.

Referring to FIG. 8 (e), the optical waveguide 50 and the substrate 30 with the LED chip 10 mounted thereon may be sealed with a light-absorbing insulator to form the insulator 70.

The insulator 70 couples the substrate 30 with the optical waveguide 50 and, at the same time, provides optical/mechanical insulation for the probe-type LED chip module 1 for biostimulation from outside. That is to say, the insulator 70 mechanically couples the probe-type LED chip module 1 for biostimulation, provides electrical insulation and waterproofing, and prevents leakage of light.

The insulator 70 may seal the probe-type LED chip module 1 for biostimulation except for the portion where the probe-type LED chip module 1 for biostimulation is connected with outside. For example, the insulator 70 may surround the probe-type LED chip module 1 for biostimulation while partly exposing the probe 55 and the electrode connector 90.

For this, the insulator 70 is formed from a biologically unharmful, highly light-absorbing, waterproofing, and electrically insulating material. That is to say, the insulator 70 may be formed from a light-absorbing insulating material, for example, black epoxy.

The method for manufacturing the probe-type LED chip module 1 for biostimulation according to the present disclosure allows for manufacturing of the probe-type LED chip module with a structure similar to that of an optical fiber as well as reduction of manufacturing cost.

Although the optical waveguide has a cylindrical structure in an exemplary embodiment of the present disclosure, the structure was selected as such for ease of manufacturing and reduction of cost. Any other structure capable of focusing light (e.g. photonic-crystal fiber, Panda/Bow-tie polarization-maintaining optical fiber, etc.) may be used.

The present disclosure is directed to providing an LED-based, small-sized, portable optical stimulation device. The optical stimulation device according to the present disclosure can replace the existing photon therapy based on large-size laser at low cost since the light source can be controlled using a portable battery.

Also, since the optical stimulation device can be manufactured at very low cost, the cost for treating cranial nerve diseases such as Parkinson's disease and depression can be decreased. In addition, since the nerves can be stimulated without restricting the activities of the living body, it may be utilized for the development of a novel medical device based on optical stimulation.

The existing laser system costs at least $50,000 and the weight of the system exceeds 300 kg including the battery. In contrast, the system according to the present disclosure costs around $200 and weighs not more than 200 g including the portable battery. A disposable probe-type LED chip module may be manufactured at a cost of around $10.

While the present disclosure has been described with respect to the specific embodiments, it will be apparent to those skilled in the art that various changes and modifications may be made without departing from the spirit and scope of the disclosure as defined in the following claims.

What is claimed is:

1. A probe-type light-emitting diode (LED) chip module for biostimulation comprising:
an LED chip;
a substrate supporting the LED chip;
an optical waveguide collecting light emitted from the LED chip; and
an insulator coupling the substrate with the optical waveguide and providing insulation from outside.

2. The probe-type LED chip module for biostimulation according to claim 1, wherein the optical waveguide comprises:
a body extending from one end facing the LED chip with a cylindrical shape;
a variable layer having a diameter decreasing gradually from the other end of the body; and
a probe extending from the end of the variable layer and having a diameter equivalent to that of an optical fiber.

3. The probe-type LED chip module for biostimulation according to claim 1, wherein the optical waveguide is formed from an optical fiber preform.

4. The probe-type LED chip module for biostimulation according to claim 3, wherein the optical waveguide is formed from silica.

5. The probe-type LED chip module for biostimulation according to claim 1, wherein the optical waveguide has a double cylindrical structure comprising:
a core formed at the center of a length direction of the optical waveguide and transmitting light emitted from the LED chip; and
a cladding surrounding the core.

6. The probe-type LED chip module for biostimulation according to claim 5, wherein the refractive index of the core of the optical waveguide is higher than the refractive index of the cladding.

7. The probe-type LED chip module for biostimulation according to claim 6, wherein the germanium oxide (GeO₂) is doped in the core of the optical waveguide.

8. The probe-type LED chip module for biostimulation according to claim 1, which further comprises an electrode connector connected to an external power supply supplying power to the LED chip.

9. The probe-type LED chip module for biostimulation according to claim 8, wherein the substrate electrically connects the LED chip with the electrode connector.

10. The probe-type LED chip module for biostimulation according to claim 9, wherein the substrate is a ceramic substrate comprising aluminum nitride (AlN) or aluminum oxide (Al₂O₃) or a printed circuit board (PCB).

11. The probe-type LED chip module for biostimulation according to claim 1, which is used for the ChR2 receptor.

12. The probe-type LED chip module for biostimulation according to claim 11, wherein the LED chip comprises a gallium nitride (GaN)-based blue LED.

13. The probe-type LED chip module for biostimulation according to claim 1, which the LED chip comprises a plurality of LEDs having different wavelength each other.

14. The probe-type LED chip module for biostimulation according to claim 1, which further comprises an optical matching material between the substrate and the optical waveguide.

15. The probe-type LED chip module for biostimulation according to claim 1, wherein the insulator is formed from a light-absorbing insulating material.

16. The probe-type LED chip module for biostimulation according to claim 15, wherein the insulator is formed from black epoxy.

17. A method for fabricating a probe-type light-emitting diode (LED) chip module for biostimulation, comprising:
extending an optical fiber preform whose refractive index at the center being higher than the refractive index at the periphery to form a cylinder-shaped intermediate preform;
extending the intermediate preform with and without heating to form an optical waveguide;
coupling the optical waveguide with a substrate on which an LED chip is mounted; and
sealing the substrate and the optical waveguide with a light-absorbing insulator.

18. The method for fabricating a probe-type LED chip module for biostimulation according to claim 17, wherein said forming the optical waveguide comprises:
extending the intermediate preform slowly with heating to form a variable layer having a diameter decreasing gradually from a portion of the intermediate preform; and
extending the end of the variable layer quickly without heating to form a probe having a diameter equivalent to that of an optical fiber.

19. The method for fabricating a probe-type LED chip module for biostimulation according to claim 17, wherein said forming the optical waveguide is performed after fixing an end of the intermediate preform to a holder.

20. The method for fabricating a probe-type LED chip module for biostimulation according to claim 17, wherein said coupling the optical waveguide with the substrate comprises injecting an optical matching material between the substrate and the optical waveguide.

21. The method for fabricating a probe-type LED chip module for biostimulation according to claim 17, wherein said coupling the optical waveguide with the substrate further comprises mirror polishing the lower end of the optical waveguide.

22. The method for fabricating a probe-type LED chip module for biostimulation according to claim 17, which further comprises forming an electrode connector connected to an external power supply supplying power at the substrate.
